(19) United States
(12) Patent Application Publication
(10) Pub. No.: US 2004/0228811 A1
(43) Pub. Date: Nov. 18, 2004

(54) Sunscreen wipes having high sunscreen formulation transfer rate

(75) Inventor: Duane G. Krzysik, Appleton, WI (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Kimberly-Clark Worldwide, Inc.

(21) Appl. No.: 10/436,774

(22) Filed: May 13, 2003

(51) Int. Cl. A61K 7/42; A61K 9/70

(52) U.S. Cl. 424/59; 424/443

(57) ABSTRACT

Sunscreen wipes comprising a thixotropic, quick-breaking sunscreen formulation are disclosed. The sunscreen formulation comprises water, at least one sunscreen active, Pemulen TR-2, a stabilizing emulsifier, and a neutralizing agent. By formulating the sunscreen formulation such that it has specific high shear and low shear viscosities at a pH range of from about 5 to about 6, the transfer rate of the sunscreen formulation to the skin during use is significantly increased as compared to conventional sunscreen wipes.

### BACKGROUND OF THE INVENTION

The present invention relates to sunscreen wipes. More particularly, the present invention relates to a wipe product comprising a thixotropic, quick-breaking sunscreen emulsion formulation formulated such that the sunscreen wipe has a high transfer rate of sunscreen formulation to the skin when used as compared to conventional sunscreen wipe products.

The undesirable effects of overexposure to sunlight are well known and understood. Such overexposure can result in not only an uncomfortable sunburn, but also in prematurely aging skin, wrinkles, loss of skin elasticity, dermatosis, and ultimately skin cancer or other serious skin problems. Sun screening is desirable in order to protect the skin from these, and other, adverse effects of solar radiation. The most dangerous solar radiation to the skin is the ultra violet (UV) radiation at wavelengths lower than about 400 nanometers, which includes both UVA and UVB radiation.

Sunscreen compositions or formulations may be applied directly to the skin to combat sunburning of the skin and the numerous problems described above. When a sunscreen formulation, utilizing the proper sun protection factor (SPF, which is a measure of the protection from the sun afforded by a sunscreen agent or composition) is applied uniformly to the body, sunscreens can be highly effective in protecting against sunburn and damage that can lead to photoaging. However, sunscreen failure can occur when areas of the body are missed during application. Typically, children are at greater risk of sunburn than adults, since coverage on children's skin tends to be more incomplete, uneven, and/or inconsistent.

Conventional sunscreen compositions are typically in the form of a liquid, either a lotion, or a cream. These compositions may be either oil or water based, and are typically emulsions. Water-based emulsions serve mainly as an aid to disperse the sunscreen active ingredients topically. Upon application to the skin, the carrier water evaporates and leaves a thin film of active ingredients plus excipient deposited on the skin. The film, which is preferably a continuous film, that remains on the skin and which contains the sunscreen actives, protects the skin from ultraviolet radiation and damage resulting therefrom. The liquid applications of these sunscreen compositions are often messy or greasy to the touch. Also, the distribution of the active ingredients on the skin may be uneven when using liquid sunscreen formulations. Also, the user must wait for the liquid in the sunscreen compositions to dry. Additionally, many conventional liquid sunscreen products are substantially non-waterproof and may require multiple applications to provide the intended sunscreen benefit over extended periods of time.

In an effort to improve upon the numerous shortcomings of liquid sunscreens, sunscreen wipes comprising a sunscreen formulation have recently become commercially available. Sunscreen wipes provide a convenient way of applying sunscreen to the body, as well as a convenient way of carrying and transporting the sunscreen product. Although sunscreen wipes are convenient and may provide a sunscreen benefit when utilized properly, conventional sunscreen wipes have to date suffered from numerous problems and shortcomings, which may decrease the overall effectiveness of the sunscreen wipe. The primary shortcoming of the sunscreen wipe has typically been a very low transfer rate; that is, conventional sunscreen wipes, when used to apply the sunscreen to the skin, actually only transfer a very small amount of the sunscreen formulation held on the wipe to the skin. Generally, only about two to three weight percent, or less, of the sunscreen formulation is actually transferred from the sunscreen wipe to the skin where it is of benefit. Such a low transfer rate can result in an insufficient amount of sunscreen being applied to the skin and a resulting sunburn.

Based on the foregoing, it is clear that there is a need in the art for a sunscreen wipe with a high transfer rate; that is, there is a need for a sunscreen wipe capable of transferring a sufficient amount of sunscreen from the wipe to the skin upon use to provide the intended sunscreen benefit. Additionally, it would be beneficial if the sunscreen formulation utilized in combination with the sunscreen wipe was substantially waterproof to reduce the need for re-application after contact with water or after extended periods of use.

### SUMMARY OF THE INVENTION

The present invention provides sunscreen wipes comprising a wipe substrate and an emulsified thixotropic, quick-breaking sunscreen formulation wherein the sunscreen wipe has a high transfer rate of sunscreen formulation from the sunscreen wipe to the skin of the user during application. Specifically, the sunscreen wipes of the present invention have a transfer rate of at least about 4% as defined herein, and preferably from about 5% to about 10%. To achieve such an improved transfer rate, the sunscreen formulations used in combination with the wipe substrate are emulsions and are formulated such that they have a low shear viscosity of from about 1000 cPs to about 10,000 cPs, a high shear viscosity of no more than about 3000 cPs, and a pH of from about 5 to about 6.

Specifically, the sunscreen formulations of the present invention comprise the following components:

(a) water;

(b) one or more sunscreen actives;

(c) Pemulen TR-2;

(d) a stabilizing emulsifier; and

(e) a neutralizing agent.

Additionally, the sunscreen formulations described herein may comprise an optional high spreadability emollient in some embodiments to increase the spreadability of the sunscreen formulation over the skin of the user. Other optional components include humectants, film formers, sunscreen boosters, vitamins, skin protectants, powders, skin conditioners, botanicals, natural extracts, alpha hydroxy acids, moisturizers, fats and oils, lipids, fatty alcohols, fatty acids, preservatives, chelating agents, antioxidants, colorants, pigments, optical brighteners, essential oils, and fragrances.

Briefly, therefore, the present invention is directed to a sunscreen wipe comprising a wipe substrate and a sunscreen formulation. The sunscreen formulation comprises from about 53.5% (by total weight of the formulation) to about 98% (by total weight of the formulation) water, from about 1% (by total weight of the formulation) to about 40% (by total weight of the formulation) sunscreen active, from about 0.1% (by total weight of the formulation) to about 0.5% (by total weight of the formulation) Pemulen TR-2, from about 0.1% (by total weight of the formulation) to about 5% (by total weight of the formulation) stabilizing emulsifier, and from about 0.1% (by total weight of the formulation) to about 1% (by total weight of the formulation) neutralizing agent. The sunscreen wipe has a transfer rate of at least about 4%.

The present invention is further directed to a sunscreen wipe comprising a wipe substrate and a sunscreen formulation. The sunscreen formulation comprises from about 53.5% (by total weight of the formulation) to about 98% (by total weight of the formulation) water, from about 1% (by total weight of the formulation) to about 40% (by total weight of the formulation) sunscreen active, from about 0.1% (by total weight of the formulation) to about 0.5% (by total weight of the formulation) Pemulen TR-2, from about 0.1% (by total weight of the formulation) to about 5% (by total weight of the formulation) stabilizing emulsifier, and from about 0.1% (by total weight of the formulation) to about 1% (by total weight of the formulation) neutralizing agent. The sunscreen wipe has a transfer rate of at least about 4%, and the sunscreen formulation has a low shear viscosity of from about 1000 cPs to about 10,000 cPs, a high shear viscosity of no more than about 3000 cPs, and a pH of from about 5 to about 6.

The present invention is further directed to a sunscreen wipe comprising a wipe substrate and a sunscreen formulation. The sunscreen formulation comprises from about 70% (by total weight of the formulation) to about 75% (by total weight of the formulation) water, about 5% (by total weight of the formulation) glycerin, from about 0.1% (by total weight of the formulation) to about 0.5% (by total weight of the formulation) Pemulen TR-2, from about 5% (by total weight of the formulation) to about 20% (by total weight of the formulation) of a high spreadability emollient, from about 7% (by total weight of the formulation) to about 12% (by total weight of the formulation) sunscreen active, from about 0.1% (by total weight of the formulation) to about 5% (by total weight of the formulation) stabilizing emulsifier, and an amount of neutralizer sufficient to adjust the pH of the sunscreen formulation to a range of from about 5.4 to about 5.6. The sunscreen wipe has a transfer rate of at least about 4%.

The present invention is further directed to a sunscreen formulation suitable for use in combination with a wipe substrate. The sunscreen formulation comprises from about 53.5% (by total weight of the formulation) to about 98.7% (by total weight of the formulation) water, from about 1% (by total weight of the formulation) to about 40% (by total weight of the formulation) sunscreen active, from about 0.1% (by total weight of the formulation) to about 0.5% (by total weight of the formulation) Pemulen TR-2, from about 0.1% (by total weight of the formulation) to about 5% (by total weight of the formulation) stabilizing emulsifier, and from about 0.1% (by total weight of the formulation) to about 1% (by total weight of the formulation) neutralizing agent. The sunscreen formulation has a pH of from about 5 to about 6.

The present invention is further directed to a sunscreen formulation suitable for use in combination with a wipe substrate. The sunscreen formulation comprises from about 53.5% (by total weight of the formulation) to about 98.2% (by total weight of the formulation) water, from about 1% (by total weight of the formulation) to about 40% (by total weight of the formulation) sunscreen active, from about 0.1% (by total weight of the formulation) to about 0.5% (by total weight of the formulation) Pemulen TR-2, from about 0.1% (by total weight of the formulation) to about 5% (by total weight of the formulation) stabilizing emulsifier, and from about 0.1% (by total weight of the formulation) to about 1% (by total weight of the formulation) neutralizing agent. The formulation has a low shear viscosity of from about 1000 cPs to about 20,000 cPs, a high shear viscosity of no more than about 3000 cPs, and a pH of from about 5 to about 6.

The present invention is further directed to a medicinal wipe comprising a wipe substrate and a medicinal formulation. The medicinal formulation comprises from about 44% (by total weight of the formulation) to about 94% (by total weight of the formulation) water, from about 0.1% (by total weight of the formulation) to about 0.5% (by total weight of the formulation) Pemulen TR-2, from about 5% (by total weight of the formulation) to about 10% (by total weight of the formulation) of a high spreadability emollient, from about 0.1% (by total weight of the formulation) to about 50% (by total weight of the formulation) medicinal active, from about 0.1% (by total weight of the formulation) to about 5% (by total weight of the formulation) stabilizing emulsifier, and an amount of neutralizing agent sufficient to adjust the pH of the formulation from about 5.4 to about 5.6. The medicinal wipe has a transfer rate of at least about 4%.

Other features and advantages of this invention will be in part apparent and in part pointed out hereinafter.

Definitions

Within the context of this specification, each term or phrase below will include, but not be limited to, the following meaning or meanings:

(a) “Bonded” refers to the joining, adhering, connecting, attaching, or the like, of two elements. Two elements will be considered to be bonded together when they are bonded directly to one another or indirectly to one another, such as when each is directly bonded to intermediate elements.

(b) “Layer” when used in the singular can have the dual meaning of a single element or a plurality of elements.

(c) “Meltblown” refers to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed for example, in U.S. Pat. No. 3,849,241 to Butin et al. Meltblown fibers are microfibers which may be continuous or discontinuous, are generally smaller than about 0.6 denier, and are generally self bonding when deposited onto a collecting surface. Meltblown fibers used in the present invention are preferably substantially continuous in length.

(d) “Nonwoven” refers to materials and webs of material which are formed without the aid of a textile weaving or knitting process.

(e) “Polymeric” includes, but is not limited to, homopolymers, copolymers, such as for example, block, graft, random and alternating copolymers, terpolymers, etc. and blends and modifications thereof. Furthermore, unless otherwise specifically limited, the term “polymeric” shall include all possible geometrical configurations of the material. These configurations include, but are not limited to, isotactic, syndiotactic and atactic symmetries.

(f) “Thermoplastic” describes a material that softens when exposed to heat and which substantially returns to a non-softened condition when cooled to room temperature.

(g) “Thixotropic” means a liquid that has a reduced viscosity when stress is applied thereto.

### DETAILED DESCRIPTION OF THE PREFERRED EMBODIMENTS

In accordance with the present invention, it has been discovered that the transfer rate of an emulsified sunscreen formulation from sunscreen wipes can be significantly improved by utilizing a thixotropic, quick-breaking sunscreen formulation. Surprisingly, when a sunscreen formulation is provided with specific low shear and high shear viscosities in combination with a specific pH, the transfer rate of the sunscreen from the wipe is dramatically improved. Also, through the use of the sunscreen wipes as described herein, users are able to apply a sufficient amount of sunscreen to ensure adequate protection, as compared to liquid sunscreens which are often under-applied resulting in skin burns.

The sunscreen wipes of the present invention comprise a wipe substrate in combination with a sunscreen formulation. The wipe substrate, or basesheet is generally rectangular in shape and may have any suitable unfolded width and length. For example, the wipe substrate may have an unfolded length of from about 2.0 centimeters to about 100.0 centimeters, and desirably from about 10.0 centimeters to about 25.0 centimeters, and an unfolded width of from about 2.0 centimeters to about 80.0 centimeters and desirably from about 10.0 centimeters to about 25.0 centimeters. Typically, each individual wipe substrate is arranged in a folded configuration and stacked one on top of the other to provide a stack of sunscreen wipes. Such folded configurations are well known to those skilled in the art and include c-folded, z-folded, quarter-folded configurations and the like. The stack of folded sunscreen wipes may be placed in the interior of a container, such as a plastic tub, to provide a package of sunscreen wipes for eventual sale to the consumer. Alternatively, the sunscreen wipes may include a continuous strip of material which has perforations between each wipe and which may be arranged in a stack or wound into a roll for dispensing.

Optionally, the sunscreen wipe may assume a variety of shapes, including but not limited to, generally circular, oval, square, or irregularly shaped depending upon numerous factors. The size of the sunscreen wipe may also vary depending upon the desired end use of the wipe.

The materials of the substrate or basesheet, whether single or multi-layered, may be varied to provide different physical properties. The different physical properties, which a layer may be configured to provide, may be controlled by selecting the appropriate materials having characteristics such as softness, resiliency, strength, flexibility, integrity, toughness, absorbency, liquid retention, thickness, tear resistance, surface texture, drapability, hand, wettability, wicking ability and the like, and combinations thereof. The sunscreen wipe substrate can be configured to provide all desired physical properties within one layer, or configured to provide only specific physical properties within individual layers of a multi-layered wipe. For example, the wipe substrate may include at least one layer of material that is configured to provide strength and resilience to the sunscreen wipe, and at least one other layer which is configured to provide a soft, gentle wiping surface to the sunscreen wipe. Desirably, the sunscreen wipes provide a soft wiping surface for contact with the skin and application of the sunscreen formulation.

The one or more layers of the sunscreen wipe product can be made from a variety of materials including meltblown materials, coform materials, air-laid materials, bonded-carded web materials, hydroentangled materials, spunbond materials and the like, and can comprise synthetic or natural fibers. Examples of natural fibers suitable for use in the present invention include cellulosic fibers such as wood pulp fibers, cotton fibers, flax fibers, jute fibers, silk fibers and the like. Examples of thermoplastic polymeric fibers suitable for use with the present invention include polyolefins such as polypropylene and polyethylene, polyamides, and polyesters such as polyethylene teraphthalate. Alternative synthetic fibers which may be suitable include staple nylon and rayon fibers. The layer or layers of the sunscreen wipe can be woven or nonwoven materials. In addition, the materials can be formed into balls, such as cotton balls, or applied to delivery systems such as applicators for swabs.

If one or more layers of the basesheet is a combination of polymeric and natural fibers, such as polypropylene and cellulosic fibers, the relative percentages of the polymeric fibers and natural fibers in the layer can vary over a wide range depending on the desired characteristics of the sunscreen wipe. For example, the layer may comprise from about 20 to about 95 weight percent, desirably from about 20 to about 60 weight percent, and more desirably from about 30 to about 40 weight percent of polymeric fibers based on the dry weight of the layer. Such a layer of polymeric and natural fibers may be manufactured by any method known to those skilled in the art.

Generally, it is desirable that a layer comprising both polymeric and natural fibers be formed by a coform process for a more uniform distribution of the polymeric and natural fibers within the layer. Such coform layers are manufactured generally as described in U.S. Pat. No. 4,100,324 to Anderson et al. which issued Jul. 11, 1978; U.S. Pat. No. 4,604,313 to McFarland et al. which issued Aug. 5, 1986; and U.S. Pat. No. 5,350,624 which issued Sep. 27, 1994; which is herein incorporated by reference to the extent they are consistent herewith.

Typically, such coform layers comprise a gas-formed matrix of thermoplastic polymeric meltblown microfibers, such as, for example, polypropylene microfibers, and cellulosic fibers, such as, for example, wood pulp fibers. A coform layer is formed by initially forming at least one primary air stream containing the synthetic or polymeric fibers and merging the primary stream with at least one secondary stream of natural or cellulosic fibers. The primary and secondary streams are merged under turbulent conditions to form an integrated stream containing a thorough, homogeneous distribution of the different fibers. The integrated air stream is directed onto a forming surface to air form the layer of material. A multiplicity of these coform layers can then be formed in succession to provide a web of multiple coform layers.

The different fibers in the different layers of the layered basesheet of the present invention, such as the polypropylene and polyethylene microfibers set forth above, typically may not be compatible with and may not bond to each other. However, the different fibers may entangle with each other resulting in suitable securement between the layers. For example, in a layered basesheet containing a coform layer of polyethylene and cellulosic fibers and a coform layer of polypropylene and cellulosic fibers, the polyethylene and polypropylene fibers may entangle with each other and the cellulosic fibers and may at least partially bond to the cellulosic fibers which results in securement between the layers. Such interlayer bonding and entanglement may be enhanced by a thermo-mechanical process wherein the layered basesheet is passed between a heated smooth anvil roll and a heated pattern roll. The pattern roll may have any raised pattern which provides the desired entanglement and interlayer bonding. Desirably, the pattern roll defines a raised pattern which defines a plurality of bond locations which define a bond area of between about 4 and about 30 percent of the total area of the roll for improved interlayer attachment.

The basesheet for the sunscreen wipe may have a total basis weight of from about 25 to about 120 grams per square meter and desirably from about 40 to about 90 grams per square meter. The basis weight of the basesheet may vary depending upon one or more desired characteristics of the sunscreen wipe. For example, a suitable basesheet for wiping sunscreen onto the skin may define a basis weight of from about 60 to about 80 grams per square meter and desirably about 75 grams per square meter. In a particular embodiment wherein the basesheet includes coform layers of polypropylene and cellulosic fibers and polyethylene and cellulosic fibers, the layered basesheet defines a basis weight of from about 60 to about 90 grams per square meter and desirably about 80 grams per square meter, for improved softness and adequate strength.

In a particular embodiment, it is desired that the sunscreen wipe of the present invention define sufficient strength to withstand the forces exerted by the user when it is used to apply the sunscreen formulation to the skin. For example, the basesheet for the sunscreen wipe may define a tensile strength of at least about 1.23 Newtons per centimeter in the machine direction and at least about 0.70 Newtons per centimeter in the cross machine direction. Sunscreen wipes having alternate ranges of tensile strength may also be effectively employed in transferring sunscreen formulation to the skin. As used herein, the term “machine direction” refers to the direction in which the material is manufactured while the cross machine direction refers to a direction which is perpendicular to the machine direction.

In a particular embodiment, wherein the basesheet includes coform layers of polypropylene and cellulosic fibers and polyethylene and cellulosic fibers, the layered basesheet defines a tensile strength of from about 1.31 to about 3.50 Newtons per centimeter in the machine direction and from about 0.84 to about 1.40 Newtons per centimeter in the cross machine direction, and desirably from about 1.58 to about 1.93 Newtons per centimeter in the machine direction and from about 0.93 to about 1.11 Newtons per centimeter in the cross machine direction. In such a configuration, the coform layer, which includes polypropylene fibers, provides the majority of the strength to the basesheet while the coform layer, which includes the polyethylene fibers, provides a soft surface for contact with the skin of the user. Thus, the tensile strength of such a layered basesheet is higher than the tensile strength of a single layer containing polyethylene fibers and polypropylene fibers.

The sunscreen formulations described herein are typically applied to the basesheet in an amount sufficient to achieve the desired transfer rates discussed herein without oversaturating the basesheet which may lead to unwanted pooling of sunscreen in the wipe container and wasted sunscreen. Specifically, the sunscreen formulation is desirably applied to the basesheet in an amount of from about 100% (by weight based on the weight of the dry basesheet) to about 1000% (by weight based on the weight of the dry basesheet), desirably from about 100% (by weight based on the weight of the dry basesheet) to about 500% (by weight based on the weight of the dry basesheet). The exact amount of sunscreen formulation applied to the basesheet may vary depending upon the desired application rate and the basesheet being utilized to deliver the sunscreen formulation. Also, the exact amount of sunscreen formulation applied to the basesheet may depend upon the packaging utilized to deliver the sunscreen wipe to the ultimate consumer. For example, if the sunscreen wipes are to be delivered in a tub comprising numerous sunscreen wipes, excess sunscreen formulation may need to be applied to the wipes to ensure coverage from the top of the tub to the bottom.

As noted above, the sunscreen wipes of the present invention include a thixotropic, quick-breaking sunscreen formulation along with the wipe substrate. This sunscreen formulation comprises numerous ingredients including sufficient water to form the sunscreen emulsion. Generally, the sunscreen formulation is from about 53.5% (by total weight of the formulation) to about 98.7% (by total weight of the formulation) water, desirably from about 60% (by total weight of the formulation) to about 80% (by total weight of the formulation), and most desirably from about 65% (by total weight of the formulation) to about 75% (by total weight of the sunscreen formulation). In some embodiments, the amount of water will be from about 73% (by total weight of the sunscreen formulation) to about 73.4% (by total weight of the sunscreen formulation). More specifically, the water may comprise 73% (by total weight of the sunscreen formulation), 73.2% (by total weight of the sunscreen formulation), or even 73.4% (by total weight of the sunscreen formulation).

Along with the water, the sunscreen formulations described herein comprise at least one sunscreen active, and desirably a combination of sunscreen actives to provide the desired sunscreen benefit. The sunscreen active may be an oil-soluble or water-soluble sunscreen active and may be a UVB sunscreen active, UVA sunscreen active, or a combination of both UVB and UVA sunscreen actives. UVA sunscreen actives protect against long wavelength actinic radiation of the sun in the 320 to 400 nanometer range and UVB sunscreen actives protect against shorter wavelength actinic radiation of the sun in the 290 to 320 nanometer range.

Sunscreen actives suitable for use in the sunscreen formulations of the present invention include, for example, benzophenone-3 (oxybenzone), benzophenone-4 (sulisobenzone), benzophenone-8 (dioxybenzone), butylmethoxydibenzoylmethane (avobenzone), cinoxate, DEA-methoxycinnamate, digalloyl trioleate, 1-(3,4-dimethoxyphenyl)-4,4-4-dimethyl-1,3-pentanediene, ethyl dihydroxypropyl PABA, ethylhexyl dimethyl PABA (padimate 0), ethylhexyl methoxycinnamate (octinozate), ethylhexyl salicylate (octisalate), 4-(2-beta-glucopyranosiloxy) propoxy-2-hydroxybenzophenone, glyceryl PABA, homosalate, mentyl anthranilate, octocrylene, PABA (aminobenzoic acid), phenylbenzimidazole sulfonic acid (ensulizole), red petrolatum, TEA salicylate, titanium dioxide, zinc oxide, surface treated titanium dioxide, surface treated zinc oxide, Spirulina Platensis Powder, Vitis Vinifera (Grape) Seed Extract, Helianthus Annus (Sunflower) Seed Extract, tocopherol, and combinations thereof. The sunscreen formulations of the present invention typically provide an SPF of from about 2 to about 50, or more.

Along with the sunscreen active or combination of sunscreen actives, a UV absorber may optionally be included to provide an additional sunscreen benefit. For example, UV absorbers such as terephthalidene dicamphor sulfonic acid, drometrizole trisoloxane, benzylylidene malonate polysiloxane, diethylhexylbutamido triazone, methylene-bis-benzotriazolyl tetramethylbutylphenol, disodium phenyl dibenzimidazole tetrasulfonate, bis-ethylhexyloxyphenol methoxyphenyl triazine, and diethylamino hydorxylbenzoyl hexyl benzoate or combinations thereof may be included in the sunscreen formulation to provide the additional benefit. Additionally, one or more of the sunscreen active or actives, and/or the UV absorbers, may be encapsulated together or separately to reduce penetration into the skin and improve the long-term effectiveness of the formulation.

The sunscreen active, or combination of actives, is included in the formulation in an amount of from about 1% (by total weight of the formulation) to about 40% (by total weight of the formulation), and desirably from about 5% (by total weight of the formulation) to about 15% (by total weight of the formulation). As will be recognized by one skilled in the art, the amount of sunscreen active and/or UV absorbers added to the sunscreen formulation may directly effect the SPF value of the sunscreen formulation.

In addition to the sunscreen active and water, because the sunscreen formulations described herein are a combination of at least two immiscible liquids (i.e., liquids that are not mutually soluble), a stabilizing emulsifier is introduced into the sunscreen formulation to create an emulsion; that is, to create a liquid that appears to be homogeneous and allows one of the immiscible liquids to remain in a continuous form, while allowing the other immiscible liquid to remain in a dispersed droplet form. Stabilizing emulsifiers typically provide two benefits: (1) assist in the production of a stable emulsion; and (2) provide a thickening or “bodying” effect to an emulsion. Typically, stabilizing emulsifiers are molecules with non-polar and polar regions that are able to reside at the interface of the two immiscible liquids. The stabilizing emulsifier will typically have a hydrophilic/lipophilic balance of from about 1 to about 7.

Suitable stabilizing emulsifiers include, for example, sorbitan monooleate, sorbitan sesquioleate, sorbitan isostearate, sorbitan trioleate, PEG-22/dodecyl glycol copolymer, PEG-45/dodecyl glycol copolymer, polyglyceryl-3-diisostearate, polyglycerol esters of oleic/isostearic acid, polyglyceryl-6 hexaricinolate, polyglyceryl-4 oleate, polyglyceryl-4 oleate/PEG-8 propylene glycol cocoate, oleamide DEA, sodium glyceryl oleate phosphate, hydrogenated vegetable glycerides phosphate, glyceryl stearate, and combinations thereof.

One or more of the stabilizing emulsifiers is introduced into the sunscreen formulation in an amount of from about 0.1% (by total weight of the formulation) to about 20% (by total weight of the formulation), desirably from about 0.1% (by weight of the formulation) to about 5% (by total weight of the formulation), and more desirably from about 0.1% (by total weight of the formulation) to about 0.5% (by total weight of the formulation).

Additionally, the sunscreen formulations of the present invention include Pemulen TR-2 (Noveon, Inc. Cleveland, Ohio) (International Nomenclature Cosmetic Ingredient name: Acrylates/C10-30 Alkyl Acrylates Crosspolymer), which is a crosslinked acrylic acid polymer containing alkyl groups, which aid in the compatibility of the overall sunscreen formulation with oily components. By introducing Pemulen TR-2 into the sunscreen formulations of the present invention, stable emulsions comprising up to about 70% oil are possible while achieving highly stable low viscosity emulsions. The sunscreen formulations described herein comprise from about 0.1% (by total weight of the formulation) to about 2% (by total weight of the formulation), desirably from about 0.1% (by total weight of the formulation) to about 0.5% (by total weight of the formulation) Pemulen TR-2.

In order for the Pemulen TR-2 to properly function in the formulation, a neutralization agent is added to the sunscreen formulation. When dispersed in water, the Pemulen TR-2 resin molecules partially swell and upon neutralization with a water soluble alkaline material, the resin molecules swell completely providing a network. This formed network holds oil droplets in place protecting each oil droplet from coalescing with another oil droplet, and thereby provides superior emulsion stability. Stated another way, upon the formation of an oil-in-water emulsion, the properly neutralized Pemulen TR-2 polymers form an adsorbed gel layer around each oil droplet, with the hydrophobic (alkyl group) portions of the polymer anchored in the oil.

Proper neutralization of the Pemulen TR-2 polymer is preferred to obtain optimal viscosity control and stability of the sunscreen emulsion. Inadequate neutralization of the Pemulen TR-2 provides emulsions with too low of a viscosity for a given use level of the polymer and/or an unstable emulsion when using low levels of the polymer. Over neutralization of the polymer can result in emulsion instability especially when the polymer is used at low levels.

Suitable neutralizing agents include, for example, sodium hydroxide, potassium hydroxide, ammonium hydroxide, sodium borate, triethanolamine, aminomethyl propanol, sodium glucamate, and the like. The neutralization agent is introduced into the sunscreen formulation to neutralize the Pemulen TR-2 until a pH of the formulation of from about 5 to about 6, desirably from about 5.4 to about 5.6 is achieved. Such a pH range not only properly neutralizes the Pemulen TR-2, but is also close to that of skin for optimum health.

In addition to viscosity control, proper neutralization of the Pemulen TR-2 provides stability of the sunscreen formulation on the basesheet, such as a woven or non-woven fiber-type basesheet as discussed above. The network formed, coupled with the hydrophobic portions (alkyl groups), hold separate the oil droplets (containing the sunscreen actives) and prevent them from preferentially being absorbed by the natural or polymer basesheet fibers.

Moreover, sunscreen emulsion formulations with properly neutralized Pemulen TR-2 have shear thinning rheology; that is, when shear is applied to the formulation, the viscosity of the formulation drops considerably. For example, when the sunscreen formulation is applied to the basesheet during manufacturing, there is shear produced by the mixers and pumps. Also, there is shear produced as the formulation is pumped through orifices (hoses and application heads) as well as velocity shear. The Pemulen TR-2 neutralized sunscreen formulations described herein shear to very low viscosities as discussed below. Upon application to the basesheet, these emulsions are thin enough from the application shear to thoroughly wet the basesheet and uniformly impregnate it. After a short period of time, the viscosity then rebounds to the original viscosity to stabilize the formulation on the basesheet. Also, the shear thinning property facilitates the transfer of the formulation to the skin. As the basesheet impregnated with the sunscreen formulation is rubbed on the skin, the sunscreen formulation thins to a lower viscosity helping the sunscreen formulation transfer more effectively to the skin.

In addition to the properties described above, the sunscreen formulations described herein, upon application to the skin, break which further facilitates transfer to the skin. This emulsion destabilization is caused by the natural salt content common on the surface of skin. The salt causes the acrylic hydrophilic portion of the Pemulen TR-2 hydrogel to instantaneously de-swell, thus releasing the oil phase or sunscreen actives and provide immediate contact of the sunscreen actives with the skin.

The sunscreen wipes of the present invention comprise a sunscreen formulation which is easily transferable from the wipe basesheet to the skin. In order to facilitate this transfer, the sunscreen formulations desirably have viscosities at low shear (an approximation of the viscosity of the product under the forces of gravity, or the formulations natural viscosity) of from about 1000 cPs to about 20,000 cPs, desirably from about 2000 cPs to about 10,000 cPs, and most desirably from about 3,000 cPs to about 6,000 cPs. These centipoise values for low shear viscosity are determined at 25° C. and a shear of 1/sec using a suitable Rheometer, such as a Rheostress Model Number RS-1 (ThermoHaake, Parmaus, N.J.). The cone and plate sensor system is used to make the viscosity measurements. Measurements of viscosity versus shear rate as described and reported herein are taken using a preprogrammed logic for viscosity versus shear rate provided by the manufacturer with the Rheometer. By having such low shear viscosities, the sunscreen formulations are highly stable on the basesheet and resist substantial migration from the surface of the basesheet to the interior of the basesheet where it is much more difficult to affect transfer to the skin.

In order to allow for the desirable shear thinning characteristics described above, the sunscreen formulations of the present invention have viscosities at high shear of no more than about 3000 cPs, desirably no more than about 2000 cPs, and most desirably no more than about 1000 cPs. These centipoise values for high shear viscosity are determined at 25° C. and a shear of 20/sec using a suitable Rheometer as described above. Such high shear viscosities impart the numerous benefits noted above regarding the shear thinning viscosity.

The sunscreen wipes of the present invention comprising the sunscreen formulations described herein have improved transfer rates of sunscreen to the skin during use as compared to conventional sunscreen wipes. As used herein, the term “transfer rate” means the amount of sunscreen formulation actually transferred from the sunscreen wipe comprising the sunscreen formulation to the user's skin. Transfer rate is reported as % T (Percent Transfer), and is calculated as follows:

% T=(X)/(T)

wherein X is the weight of sunscreen formulation on the sunscreen wipe in milligrams/centimeter squared, and T is the weight of sunscreen actually transferred from the wipe to the skin or substrate, reported in grams/centimeter squared.

In accordance with the present invention, the transfer rate of the sunscreen wipes is at least about 4%, desirably from at least about 4% to about 15%, and most desirably from about 5% to about 10%. Transfer rates in these ranges are a substantial improvement over the transfer rates of conventional sunscreen wipes.

Because many sunscreen actives are viscous, oily feeling, and tacky to the touch, they typically do not spread well or evenly upon application to the skin. In one embodiment of the present invention, an optional high spreading emollient may be added to the sunscreen formulation to increase the spreadability of the sunscreen formulation over the skin. The addition of a high spreadability emollient facilitates the rub-in of the sunscreen on the skin and improves the slip of the sunscreen wipe comprising the sunscreen during application. In accordance with the present invention, suitable high spreadability emollients include those emollients having a spreading coefficient greater than zero. Spreading coefficient (S), which is the measure of the tendency of a liquid to spread is defined as:

S=Wₐ−W_{c}=γ_{w}−γₒ−γ_{w/o}

wherein Wₐ is the work of adhesion between and oil and water, W_{c} is the work of cohesion in the less dense liquid, γ_{w} is the surface tension of water (72 dynes/cm at 25° C.), γₒ is the surface tension of an oily liquid and γ_{w/o} is the interfacial tension between water and the oil. The spreading coefficient, surface tension, and interfacial tension are all defined in dynes/cm at about 25° C. The spreading coefficient predicts that spreading of the emollient will occur when S is greater than zero and spreading of the emollient will not occur when S is less than zero. The higher the value (positive), the more spreadable the emollient.

Suitable high spreadability emollients include, for example, dimethicone, cyclomethicone, C12-15 alkyl benzoates, isopropyl palmitate, isopropyl myristate, isopropyl benzoate, diisopropyl adipate, isostearyl benzoate, octyl benzoate, dipropylene glycol dibenzoate, octyidodecyl benzoate, cetearyl octanoate, C12-15 alkyl octanoate, octyl dodecanol, dimethyl capramide, butyloctyl salicyclate, diisoamyl malate, and combinations thereof. The high spreadability emollient may be introduced into the sunscreen formulation in an amount of from about 0.5% (by total weight of the formulation) to about 15% (by total weight of the formulation.

These high spreadability emollients improve the overall sunscreen formulation by allowing it to more easily spread across the skin's surface. Based on the disclosure herein, one skilled in the art will recognize that the oil phase of the emulsion should not be too spreadable; that is, the oil phase of the emulsion should not be too thin. Too thin of an oil film of sunscreen actives will lower the SPF of the sunscreen formulation. As such, the sunscreen formulation should be balanced using the proper level and type of high spreadability emollient in combination with the particular sunscreen actives and levels to ensure the proper combination of rub in, slip, and uniform film application.

Another ingredient that may optionally be added to the sunscreen formulation in some embodiments of the present invention is humectants. A humectant is a moistening agent that promotes retention of water due to its hydroscopic properties, and moisturizes the skin. One or more humectants may be introduced into the sunscreen formulation in an amount of from about 1% (by total weight of the formulation) to about 10% (by total weight of the formulation).

Suitable humectants for inclusion in the sunscreen formulations described herein include, for example, glycerin, sorbitol, polyols, sugars, hydrogenated starch hydrolysates, salts of PCA, lactates, and urea. A particularly preferred humectant is glycerin.

Another optional ingredient that may be introduced into the sunscreen formulations of the present invention is film formers or waterproofing agents. Film formers may be added to the sunscreen formulation to improve the water resistance or water proofing of the sunscreen formulation such that the sunscreen formulation will remain on the skin for a longer period of time after exposure to water. By using a suitable film former, the need for re-application of the sunscreen over time may be reduced. Examples of suitable film formers include, for example, PVM/MA decadiene crosspolymer, PVP/ecosien copolymer, polydimethylsiloxanes, and organo-polydimethyl siloxanes.

Another optional ingredient that may be included in the sunscreen formulations described herein is sunscreen boosters. Sunscreen boosters are cosmetic-type materials that have a low level SPF value which are used to boost the overall SPF in sunscreen formulations. Suitable sunscreen boosters include, for example, phenyl siloxanes, tocopherol, tocopherol esters, and Sun-Spheres®.

A dry-feel modifier can also optionally be added to the sunscreen formulations described herein. A dry-feel modifier is a compound which, when added to an emulsion, imparts a “dry feel” to the skin when the emulsion dries on the skin surface. Dry-feel modifiers may also reduce sunscreen migration on the skin surface. Suitable dry-feel modifiers include, for example, starches, talc, kaolin, chalk, zinc oxide, polydimethylsiloxanes, organo-modified polydimethyl siloxanes, silicone gums, silicone resins, inorganic salts such as barium sulfate and sodium chloride, C6 to C12 alcohols such as octanol, sulfonated oils, surface treated silica, precipitated silica, and fumed silica.

A preservative, such as an antimicrobial preservative, can also be optionally added to the sunscreen formulations as described herein. An antimicrobial preservative is a compound or substance that destroys, prevents, or inhibits the multiplication/growth of microorganisms in the sunscreen composition and may offer some protection from oxidation of the sunscreen formulation. Preservatives are used to make self-sterilizing, aqueous-based products such as emulsions. Suitable preservatives include, for example, the lower alkyl esters of para-hydroxybenzoates such as methylparaben, propylparaben, isobutylparaben, and mixtures thereof, benzyl alcohol, DMDM Hydantoin, and benzoic acid.

Another optional ingredient that can be added to the sunscreen formulations described herein is antioxidants. An antioxidant is a natural or synthetic substance added to the sunscreen formulation to protect it from decay or deterioration due to the reaction with oxygen in the air. Antioxidants prevent deterioration which may lead to the generation of rancidity and non-enzymatic browning reaction products. Suitable antioxidants include, for example, propyl, octyl and dodecyl esters of gallic acid, butylated hydroxyanisole, butylated hydroxytoluene, nordihydroguaiaretic acid, vitamin E, vitamin E acetate, vitamin C, lipoic acid, ubiquinone, Apple Green Tea, Arnica Special, Avocado GW, Bell Pepper Fruit, Black Currant B, Black Currant Green Tea, Blueberry Fruit, Cabbage Rose Extract, Camellia sinensis, Canadian Willowherb, Carrot Root, Camellia Oleifera Extract, Common thyme, Cranberry Green Tea, Echinacea Dry Aqueous Extract, Fennel Fruit, Ginkgo Biloba, Glycine Max (soybean seed), Goldenseal, Grapefruit, Grape Seed Extract and constituents thereof (proanthocyanidins), Grapefruit Green Tea, Green Tea, catechin constituents of Green Tea that include epigallocatechin gallate, epicathechin gallate, Green Tea Extra, Green Tea HS, Lime Blossom, Orange Green Tea, Rosemary Plant, Sea Parsley, St. John's Wort W/S, Strawberry Fruit, Tomato Root, Turkish Oregano, Wheat Seed, White Mistle Toe, White Tea, Yarrow, Yucca 70, Yucca Extract Powder, and combinations thereof.

Another optional ingredient that can be included in the sunscreen formulation is chelating agents. Chelating agents are compounds used to chelate or bind metallic ions with a certain heterocyclic ring structure so that the ion is held by chemical bonds from each of the participating ring. Chelating agents may be used to bind metals found in the formulation water which can catalyze various reactions causing formulation instability. Suitable chelating agents include, for example, ethylene diaminetetraacetic acid (EDTA), EDTA disodium, calcium disodium edetate, EDTA trisodium EDTA tetrasodium and EDTA dipotassium, desferal and diethylenetriaminepentaacetate (DTPA).

Fragrances can also be added to the sunscreen formulations described herein. Fragrances are aromatic compounds which can impart an aesthetically pleasing aroma to the sunscreen composition. Typical fragrances include, for example, aromatic materials extracted from botanical sources such as rose petals, gardenia blossoms, and jasmine flowers which can be used alone or in any combination to create essential oils. Additionally, alcoholic extracts may be prepared for fragrances.

Additional optional ingredients which can be incorporated into the sunscreen formulation include vitamins, skin protectants, powders, skin conditioners, botanicals, natural extracts, alpha hydroxy acids, moisturizers, fats and oils, lipids, fatty alcohols, fatty acids, colorants, pigments, optical brighteners, and essential oils.

The sunscreen wipes of the present invention can be packaged in any manner sufficient to protect the sunscreen wipe from premature degredation while allowing for easy use by the consumer. In a preferred packaging embodiment, the sunscreen wipes are packaged individually in vacuum sealed packets to prevent oxidation of one or more wipe components and prolong shelf life.

In another embodiment of the present invention, the sunscreen formulation used in combination with the wipe substrate is replaced with a medicinal formulation to provide a medicinal wipe comprised of a wipe substrate and a medicinal formulation. The medicinal wipe, similar to the sunscreen wipe, has a high degree of transfer from the wipe substrate to the skin upon use. The wipe substrate is the same wipe substrate suitable for use in combination with the sunscreen formulation as described above.

The medicinal formulation for use in combination with the wipe substrate comprises from about 44% (by total weight of the formulation) to about 94% (by total weight of the formulation) water, from about 0.1% (by total weight of the formulation) to about 0.5% (by total weight of the formulation) Pemulen TR-2, from about 5% (by total weight of the formulation) to about 10% (by total weight of the formulation) of a high spreadability emollient, from about 0.1% (by total weight of the formulation) to about 50% (by total weight of the formulation) medicinal active, from about 0.1% (by total weight of the formulation) to about 5% (by total weight of the formulation) stabilizing emulsifier, and an amount of neutralizing agent sufficient to adjust the pH of the formulation from about 5.4 to about 5.6. The water, Pemulen TR-2, high spreadability emollient, stabilizing emulsifier and neutralizing agent are suitably the same as used in combination with the sunscreen formulation and described above. Additionally, the optional ingredients described above for optional use in combination with the sunscreen formulation are suitable optional ingredients for use in combination with the medicinal formulations as well.

Numerous medicinal actives are suitable for use in the medicinal formulations described herein. In one embodiment, the medicinal active is a skin protectant which is used to protect the skin from various environmental challenges. Suitable skin protectants include calamine in an amount of from about 1% (by total weight of the formulation) to about 25% (by total weight of the formulation), cocoa butter in an amount of from about 1% (by total weight of the formulation) to about 50% (by total weight of the formulation), petrolatum in an amount of from about 30% (by total weight of the formulation) to about 50% (by total weight of the formulation), zinc oxide in an amount of from about 1% (by total weight of the formulation) to about 25% (by total weight of the formulation), lanolin in an amount of from about 1% (by total weight of the formulation) to about 20% (by total weight of the formulation) and witch hazel in an amount of from about 1% (by total weight of the formulation) to about 50% (by total weight of the formulation).

Another suitable medicinal active for use in the medicinal formulations is a psoriasis medication. Suitable psoriasis medications include pyrithone zinc in an amount of from about 0.1% (by total weight of the formulation) to about 0.25% (by total weight of the formulation), salicylic acid in an amount of from about 1% (by total weight of the formulation) to about 3% (by total weight of the formulation), and selenium sulfide in an amount of about 1%.

Another suitable medicinal active for use in the medicinal formulations is a antimicrobial active. Suitable antimicrobials include bacitracin, chlortetrcycline hydrochloride, neomycin sulfate, and tetracycline hydrochloride.

Another suitable medicinal active for use in the medicinal formulations is an antifungal. Suitable antifungals include cliquinol in an amount of about 3% (by total weight of the formulation), haloprogin in an amount of about 1% (by total weight of the formulation), miconazole nitrate in an amount of about 2% (by total weight of the formulation), povidone iodine in an amount of about 10% (by total weight of the formulation), and tolnaftate in an amount of about 1% (by total weight of the formulation).

Another suitable medicinal active for use in the medicinal formulations is an anorectal drug. Suitable anorectal drugs include benzocaine in an amount of from about 5% (by total weight of the formulation) to about 20% (by total weight of the formulation), benzyl alcohol in an amount of from about 1% (by total weight of the formulation) to about 4% (by total weight of the formulation), dibucaine in an amount of from about 0.25% (by total weight of the formulation) to about 1% (by total weight of the formulation), lidocaine in an amount of from about 2% (by total weight of the formulation) to about 5% (by total weight of the formulation) and tetracaine in an amount of from about 0.5% (by total weight of the formulation) to about 1% (by total weight of the formulation).

Another suitable medicinal active for use in the medicinal formulations is an acne medication. Suitable acne medications include salicylic acid in an amount of from about 0.5% (by total weight of the formulation) to about 2% (by total weight of the formulation) and Benzoyl peroxide in an amount of from about 2% (by total weight of the formulation) to about 10% (by total weight of the formulation).

The medicinal wipes of the present invention comprise a medicinal formulation which is easily transferable from the wipe basesheet to the skin. In order to facilitate this transfer, the medicinal formulations desirably have viscosities at low shear (an approximation of the viscosity of the product under the forces of gravity, or the formulations natural viscosity) of from about 1000 cPs to about 20,000 cPs, desirably from about 2000 cPs to about 10,000 cPs, and most desirably from about 3,000 cPs to about 6,000 cPs. These centipoise values for low shear viscosity are determined at 25° C. and a shear of 1/sec using a suitable Rheometer, such as a Rheostress Model Number RS-1 (ThermoHaake, Parmaus, N.J.). The cone and plate sensor system is used to make the viscosity measurements. Measurements of viscosity versus shear rate as described and reported herein are taken using a preprogrammed logic for viscosity versus shear rate provided by the manufacturer with the Rheometer. By having such low shear viscosities, the medicinal formulations are highly stable on the basesheet and resist substantial migration from the surface of the basesheet to the interior of the basesheet where it is much more difficult to effect transfer to the skin.

In order to allow for the desirable shear thinning characteristics described above, the medicinal formulations of the present invention have viscosities at high shear of no more than about 3000 cPs, desirably no more than about 2000 cPs, and most desirably no more than about 1000 cPs. These centipoise values for high shear viscosity are determined at 25° C. and a shear of 20/sec using a suitable Rheometer as described above. Such high shear viscosities impart the numerous benefits noted above regarding the shear thinning viscosity.

In accordance with the present invention, the transfer rate of the medicinal wipes is at least about 4%, desirably from at least about 4% to about 15%, and most desirably from about 5% to about 10%. Transfer rates in these ranges are a substantial improvement over the transfer rates of conventional medicinal wipes.

The medicinal formulations described herein are typically applied to the basesheet in an amount sufficient to achieve the desired transfer rates discussed herein without oversaturating the basesheet which may lead to unwanted pooling of medicinal formulation in the wipe container and wasted formulation. Specifically, the medicinal formulations are desirably applied to the basesheet in an amount of from about 100% (by weight based on the weight of the dry basesheet) to about 1000% (by weight based on the weight of the dry basesheet), desirably from about 100% (by weight based on the weight of the dry basesheet) to about 500% (by weight based on the weight of the dry basesheet). The exact amount of medicinal formulation applied to the basesheet may vary depending upon the desired application rate and the basesheet being utilized to deliver the medicinal formulation. Also, the exact amount of medicinal formulation applied to the basesheet may depend upon the packaging utilized to deliver the medicinal wipe to the ultimate consumer. For example, if the medicinal wipes are to be delivered in a tub comprising numerous medicinal wipes, excess medicinal formulation may need to be applied to the wipes to ensure coverage from the top of the tub to the bottom.

### EXAMPLE 1

In this Example, two separate batches of the same sunscreen formulation were prepared and tested (10 replicates of each batch) utilizing an ink rub instrument to determine the amount of sunscreen transfer from a basesheet to a collagen material receiver substrate.

Each of the two batches of sunscreen formulation were comprised as set forth in Table 1:

TABLE 1

| | Weight % (Based on Total |
|---|---|
| Ingredient | Formulation Weight) |
| WATER PHASE | |
| Water | 64.75 |
| Glycerin | 5.0 |
| Triethanolamine | 0.1 |
| Methyl Paraben | 0.15 |
| DMDM Hydatoin | 1.0 |
| OIL PHASE | |
| C12-C15 Alkyl Benzoate | 3.0 |
| Butyl Methoxydibenzoylmethane | 3.0 |
| Octyl Methoxycinnamate | 7.5 |
| Mentyl Anthranilate | 5.0 |
| Octyl Salicylate | 5.0 |
| Sorbitan Monoleate | 0.1 |
| Dow Corning 245 Fluid | 5.0 |
| Pina Colada II | 0.25 |
| Pemulen TR-2 | 0.15 |

The high shear viscosity, low shear viscosity, and pH of batch number 1 and batch number 2 are shown in Table 2.

TABLE 2

| | Low Shear Viscosity | High Shear Viscosity | pH @ |
|---|---|---|---|
| Formulation | 1 (1/sec) @ 25° C. | 20 (1/sec) @ 25° C. | 25° C. |
| Batch #1 | 1989 | 443 | 5.4 |
| Batch #2 | 2389 | 543 | 5.5 |

Each sunscreen formulation was introduced onto a coform basesheet at the add on rate of about 0.027 grams/centimeter squared. The sunscreen formulation was introduced onto the coform basesheet utilizing an Atlas Laboratory Wringer Type LW-1 instrument (Atlas Electric Devices Co., Chicago Ill.). The sunscreen formulation was placed on the moving Atlas Wringer rollers, and the basesheet run through the rollers which yielded a substantially uniform saturation of the basesheet. The amount of sunscreen formulation applied to the basesheet was controlled by the pressure applied to the rollers. The amount of sunscreen formulation applied to the basesheet was about 10 grams.

The ink rub instrument utilized was an Ink Rub Tester, Model Number 10-18-01-0001 (Testing Machines Inc., Islandia N.Y.). The ink rub method functions by rubbing a block, covered with a receptor material, against a stable base covered by the treated or coated basesheet.

Preparation of Testing Materials For Ink Rub Instrument

The collagen film receptor material was weighed and the test substrate, comprising the formulation to be tested, was cut to a dimension of about 19.2 cm by about 9.5 cm.

Testing Procedure

A four pound weighted block was used during the testing. The weighted block was covered with standard aluminum foil to prevent potential contamination of the sunscreen formulation due to migration through the collagen receptor film. The receptor material was then placed onto the weighted block with the rough side facing downward. The receptor material was held to the weighted block with four magnets. Tension across the face of the receptor material was uniform and there were no gaps between the material and the block. The sunscreen wipe was centered upon the rubber pad, and the ends of the wipe pressed on the Velcro to secure the material in place.

The ink-rub tester was set for ten cycles, where one cycle is one back and forth motion of the weighted block. After the 10 cycles were complete, the sunscreen wipe was discarded, and the collagen film was carefully removed and weighed. The change in weight of the collage film was defined as the difference between the initial and final weight of the film, and was representative of the amount of sunscreen formulation transferred from the sunscreen wipe to the collagen film. The percent transfer was calculated according to the following equation:

% Transfer=(X)/(T)

wherein X was the weight (add-on) of the sunscreen formulation on the sunscreen wipe in milligrams/centimeter squared and T was the weight of the sunscreen actually transferred from the sunscreen wipe to the collagen film, reported in milligrams/centimeter squared. The average percent transfer for each block, along with standard deviation, is shown in Table 3.

TABLE 3

| | Transfer | Standard | | Standard |
|---|---|---|---|---|
| Formulation | (mg/cm²) | Deviation | % Transfer | Deviation |
| Batch #1 | 0.14 | 0.02 | 5.2 | 0.70 |
| Batch #2 | 0.14 | 0.02 | 5.1 | 0.80 |

As the data in Table 3 indicate, both batches of sunscreen formulations transferred more than 5% of the sunscreen formulation from the basesheet to the collagen receiver substrate. Such a rate of transfer represent a significant improvement as compared to conventional sunscreen wipes.

### EXAMPLE 2

In this Example, sunscreen formulations were made comprising three different concentrations of Pemulen TR-2 and evaluated for viscosity and shear thinning at various shear rates (at 25° C.).

The composition of sunscreen formulations 1, 2, and 3 are shown in Table 4 below. The percentages of each component are given in weight percent based on the total weight of the sunscreen formulation.

TABLE 4

| Component | Formulation 1 | Formulation 2 | Formulation 3 |
|---|---|---|---|
| Water | 73.4% | 73.2% | 73% |
| Glycerin | 5.0% | 5.0% | 5.0% |
| Pemulen TR-2 | 0.1% | 0.3% | 0.5% |
| Finsolv TN | 5.5% | 5.5% | 5.5% |
| Octyl | 7.5% | 7.5% | 7.5% |
| Methoxycinnamate | | | |
| Oxybenzone | 3.0% | 3.0% | 3.0% |
| Sorbitan Oleate | 0.2% | 0.2% | 0.2% |
| Fragrance Pina | 0.3% | 0.3% | 0.3% |
| Colada | | | |
| Cyclomethicone | 5.0% | 5.0% | 5.0% |
| Triethanolamine | pH = 5.5 | pH = 5.5 | pH = 5.5 |

Each formulation was adjusted to a pH of 5.5 as noted in Table 4. The initial viscosity (1 1/sec) for formulation 1, 2, and 3 was 2472 cPs, 9343 cPs, and 31180 cPs, respectively. The results of the viscosity measurements at different rates if shear are given in Table 5 and shown graphically in FIG. 1.

TABLE 5

| Shear (1/sec) | Formulation #1 | Formulation #2 | Formulation #3 |
|---|---|---|---|
| 5 | 992 | 3148 | 5829 |
| 10 | 708 | 2150 | 3711 |
| 15 | 535 | 1549 | 2587 |
| 20 | 470 | 1332 | 2210 |
| 25 | 403 | 1123 | 1856 |
| 30 | 370 | 1026 | 1691 |

The data in Table 5 and FIG. 1 indicate that each of the formulations are highly shear thinning, which is important for sunscreen formulations for at least two reasons: (1) during application of the sunscreen formulation to a basesheet, the formulation shear thins to a viscosity that makes it easily absorbed by the basesheet; because the formulations are thixotropic, the formulation viscosity returns to the initial viscosity after application to become highly stationary on the basesheet; and (2) when the basesheet comprising the formulation is wiped across the skin during use, the shear forces of rubbing shear thins the formulation which enables a high rate of transfer.

In view of the above, it will be seen that the several objects of the invention are achieved. As various changes could be made in the above-described wound management products without departing from the scope of the invention, it is intended that all matter contained in the above description be interpreted as illustrative and not in a limiting sense.

What is claimed is:

1. A sunscreen wipe comprising a wipe substrate and a sunscreen formulation, the sunscreen formulation comprising from about 53.5% (by total weight of the formulation) to about 98.7% (by total weight of the formulation) water, from about 1% (by total weight of the formulation) to about 40% (by total weight of the formulation) sunscreen active, from about 0.1% (by total weight of the formulation) to about 0.5% (by total weight of the formulation) Pemulen TR-2, from about 0.1% (by total weight of the formulation) to about 5% (by total weight of the formulation) stabilizing emulsifier, and from about 0.1% (by total weight of the formulation) to about 1% (by total weight of the formulation) neutralizing agent, wherein the sunscreen wipe has a transfer rate of at least about 4%.

2. The sunscreen wipe as set forth in claim 1 wherein the transfer rate is from at least about 4% to about 15%.

3. The sunscreen wipe as set forth in claim 1 wherein the transfer rate is from about 5% to about 10%.

4. The sunscreen wipe as set forth in claim 1 wherein the sunscreen formulation has a low shear viscosity of from about 1000 cPs to about 20,000 cPs.

5. The sunscreen wipe as set forth in claim 1 wherein the sunscreen formulation has a low shear viscosity of from about 2000 cPs to about 10,000 cPs.

6. The sunscreen wipe as set forth in claim 1 wherein the sunscreen formulation has a low shear viscosity of from about 3000 cPs to about 6,000 cPs.

7. The sunscreen wipe as set forth in claim 1 wherein the sunscreen formulation has a high shear viscosity of no more than about 3000 cPs.

8. The sunscreen wipe as set forth in claim 1 wherein the sunscreen formulation has a high shear viscosity of no more than about 2000 cPs.

9. The sunscreen wipe as set forth in claim 1 wherein the sunscreen formulation has a high shear viscosity of no more than about 1000 cPs.

10. The sunscreen wipe as set forth in claim 1 wherein the sunscreen formulation has a pH of from about 5 to about 6.

11. The sunscreen wipe as set forth in claim 1 wherein the sunscreen formulation has a pH of from about 5.4 to about 5.6.

12. The sunscreen wipe as set forth in claim 1 wherein the sunscreen active is selected from the group consisting of benzophenone-3 (oxybenzone), benzophenone-4 (sulisobenzone), benzophenone-8 (dioxybenzone), butylmethoxydibenzoylmethane (avobenzone), cinoxate, DEA-methoxycinnamate, digalloyl trioleate, 1-(3,4-dimethoxyphenyl)-4,4-4-dimethyl-1,3-pentanediene, ethyl dihydroxypropyl PABA, ethylhexyl dimethyl PABA (padimate 0), ethylhexyl methoxycinnamate (octinozate), ethylhexyl salicylate (octisalate), 4-(2-beta-glucopyranosiloxy) propoxy-2-hydroxybenzophenone, glyceryl PABA, homosalate, mentyl anthranilate, octocrylene, PABA (aminobenzoic acid), phenylbenzimidazole sulfonic acid (ensulizole), red petrolatum, TEA salicylate, titanium dioxide, zinc oxide, surface treated titanium dioxide, surface treated zinc oxide, Spirulina Platensis Powder, Vitis Vinifera (Grape) Seed Extract, Helianthus Annus (Sunflower) Seed Extract, tocopherol, and combinations thereof.

13. The sunscreen wipe as set forth in claim 1 wherein the sunscreen active is encapsulated.

14. The sunscreen wipe as set forth in claim 1 wherein the sunscreen active comprises a UV absorber selected from the group consisting of terephthalidene dicamphor sulfonic acid, drometrizole trisiloxane, benzylylidene malonate polysiloxane, diethylhexylbutamido triazone, methylene-bis-benzotriazolyl tetermethylbutylphenol, disodium phenyl dibenzimidazole tetersulfonate, bis-ethylhexyloxyphenol methoxyphenyl triazine, diethylamino hydroxylbenzoly hexyl benzoate, and combinations thereof.

15. The sunscreen wipe as set forth in claim 1 wherein the stabilizing emulsifier has a hydrophilic/lipophilic balance of from about 1 to about 7.

16. The sunscreen wipe as set forth in claim 1 wherein the stabilizing emulsifier is selected from the group consisting of sorbitan monooleate, sorbitan sesquioleate, sorbitan isostearate, sorbitan trioleate, PEG-22/dodecyl glycol copolymer, PEG-45/dodecyl glycol copolymer, polyglyceryl-3-diisostearate, polyglycerol esters of oleic/isostearic acid, polyglyceryl-6 hexaricinolate, polyglyceryl-4 oleate, polyglyceryl-4 oleate/PEG-8 propylene glycol cocoate, oleamide DEA, sodium glyceryl oleate phosphate, hydrogenated vegetable glycerides phosphate, glyceryl stearate, and combinations thereof.

17. The sunscreen wipe as set forth in claim 1 wherein the neutralizing agent is a base.

18. The sunscreen wipe as set forth in claim 1 wherein the neutralizing agent is triethanolamine.

19. The sunscreen wipe as set forth in claim 1 wherein the neutralizing agent is sodium borate.

20. The sunscreen wipe as set forth in claim 1 wherein the sunscreen formulation further comprises from about 0.5% (by total weight of the formulation) to about 15% (by total weight of the formulation) of a high spreadability emollient.

21. The sunscreen wipe as set forth in claim 20 wherein the high spreadability emollient is selected from the group consisting of dimethicone, cyclomethicone, C12-15 alkyl benzoates, isopropyl palmitate, isopropyl myristate, isopropyl benzoate, diisopropyl adipate, isostearyl benzoate, octyl benzoate, dipropylene glycol dibenzoate, octyldodecyl benzoate, cetearyl octanoate, C12-15 alkyl octanoate, octyl dodecanol, dimethyl capramide, butyloctyl salicylate, diisoamyl malate, and combinations thereof.

22. The sunscreen wipe as set forth in claim 1 further comprising from about 1% (by total weight of the formulation) to about 10% (by total weight of the formulation) of a humectant.

23. The sunscreen wipe as set forth in claim 22 wherein the humectant is selected from the group consisting of glycerin, sorbitol, polyols, sugars, hydrogenated starch hydrolysates, salts of PCA, lactates, and urea.

24. The sunscreen wipe as set forth in claim 1 further comprising an additional ingredient selected from the group consisting of film formers, sunscreen boosters, vitamins, skin protectants, powders, skin conditioners, botanicals, natural extracts, alpha hydroxy acids, moisturizers, fats and oils, lipids, fatty alcohols, fatty acids, preservatives, chelating agents, antioxidants, colorants, pigments, optical brighteners, essential oils, dry-feel modifiers, and fragrances.

25. The sunscreen wipe as set forth in claim 1 wherein the wipe substrate is comprised of a material selected from the group consisting of meltblown, coform, air-laid, bonded-carded web, hydroentangled, and spunbond.

26. The sunscreen wipe as set forth in claim 25 wherein the material is comprised of synthetic or natural fibers.

27. A sunscreen wipe comprising a wipe substrate and a sunscreen formulation, the sunscreen formulation comprising from about 53.5% (by total weight of the formulation) to about 98.2% (by total weight of the formulation) water, from about 1% (by total weight of the formulation) to about 40% (by total weight of the formulation) sunscreen active, from about 0.1% (by total weight of the formulation) to about 0.5% (by total weight of the formulation) Pemulen TR-2, from about 0.1% (by total weight of the formulation) to about 5% (by total weight of the formulation) stabilizing emulsifier, and from about 0.1% (by total weight of the formulation) to about 1% (by total weight of the formulation) neutralizing agent, wherein the sunscreen wipe has a transfer rate of at least about 4% and the formulation has a low shear viscosity of from about 1000 cPs to about 20,000 cPs, a high shear viscosity of no more than about 3000 cPs, and a pH of from about 5 to about 6.

28. The sunscreen wipe as set forth in claim 27 wherein the transfer rate is from about least about 4% to about 15%.

29. The sunscreen wipe as set forth in claim 27 wherein the transfer rate is from about 5% to about 10%.

30. The sunscreen wipe as set forth in claim 27 wherein the sunscreen formulation has a low shear viscosity of from about 2000 cPs to about 10,000 cPs.

31. The sunscreen wipe as set forth in claim 27 wherein the sunscreen formulation has a low shear viscosity of from about 3000 cPs to about 6,000 cPs.

32. The sunscreen wipe as set forth in claim 27 wherein the sunscreen formulation has a high shear viscosity of no more than about 2000 cPs.

33. The sunscreen wipe as set forth in claim 27 wherein the sunscreen formulation has a high shear viscosity of no more than about 1000 cPs.

34. The sunscreen wipe as set forth in claim 27 wherein the sunscreen formulation has a pH of from about 5.4 to about 5.6.

35. The sunscreen wipe as set forth in claim 27 wherein the sunscreen active is selected from the group consisting of benzophenone-3 (oxybenzone), benzophenone-4 (sulisobenzone), benzophenone-8 (dioxybenzone), butylmethoxydibenzoylmethane (avobenzone), cinoxate, DEA-methoxycinnamate, digalloyl trioleate, 1-(3,4-dimethoxyphenyl)-4,4-4-dimethyl-1,3-pentanediene, ethyl dihydroxypropyl PABA, ethylhexyl dimethyl PABA (padimate 0), ethylhexyl methoxycinnamate (octinozate), ethylhexyl salicylate (octisalate), 4-(2-beta-glucopyranosiloxy) propoxy-2-hydroxybenzophenone, glyceryl PABA, homosalate, mentyl anthranilate, octocrylene, PABA (aminobenzoic acid), phenylbenzimidazole sulfonic acid (ensulizole), red petrolatum, TEA salicylate, titanium dioxide, zinc oxide, surface treated titanium dioxide, surface treated zinc oxide, Spirulina Platensis Powder, Vitis Vinifera (Grape) Seed Extract, Helianthus Annus (Sunflower) Seed Extract, tocopherol, and combinations thereof.

36. The sunscreen wipe as set forth in claim 27 wherein the sunscreen active is encapsulated.

37. The sunscreen wipe as set forth in claim 27 wherein the sunscreen active comprises a UV absorber selected from the group consisting of terephthalidene dicamphor sulfonic acid, drometrizole trisiloxane, benzylylidene malonate polysiloxane, diethylhexylbutamido triazone, methylene-bis-benzotriazolyl tetermethylbutylphenol, disodium pheny dibenzimidazole tetersulfonate, bis-ethylhexyloxyphenol methoxyphenyl triazine, diethylamino hydroxylbenzoly hexyl benzoate, and combinations thereof.

38. The sunscreen wipe as set forth in claim 27 wherein the stabilizing emulsifier has a hydrophilic/lipophilic balance of from about 1 to about 7.

39. The sunscreen wipe as set forth in claim 27 wherein the stabilizing emulsifier is selected from the group consisting of sorbitan monooleate, sorbitan sesquioleate, sorbitan isostearate, sorbitan trioleate, PEG-22/dodecyl glycol copolymer, PEG-45/dodecyl glycol copolymer, polyglyceryl-3-diisostearate, polyglycerol esters of oleic/isostearic acid, polyglyceryl-6 hexaricinolate, polyglyceryl-4 oleate, polyglyceryl-4 oleate/PEG-8 propylene glycol cocoate, oleamide DEA, sodium glyceryl oleate phosphate, hydrogenated vegetable glycerides phosphate, glyceryl stearate, and combinations thereof.

40. The sunscreen wipe as set forth in claim 27 wherein the neutralizing agent is a base.

41. The sunscreen wipe as set forth in claim 27 wherein the neutralizing agent is triethanolamine.

42. The sunscreen wipe as set forth in claim 27 wherein the neutralizing agent is sodium borate.

43. The sunscreen wipe as set forth in claim 27 wherein the sunscreen formulation further comprises from about 0.5% (by total weight of the formulation) to about 15% (by total weight of the formulation) of a high spreadability emollient.

44. The sunscreen wipe as set forth in claim 43 wherein the high spreadability emollient is selected from the group consisting of dimethicone, cyclomethicone, C12-15 alkyl benzoates, isopropyl palmitate, isopropyl myristate, isopropyl benzoate, diisopropyl adipate, isostearyl benzoate, octyl benzoate, dipropylene glycol dibenzoate, octyldodecyl benzoate, cetearyl octanoate, C12-15 alkyl octanoate, octyl dodecanol, dimethyl capramide, butyloctyl salicylate, diisoamyl malate, and combinations thereof.

45. The sunscreen wipe as set forth in claim 27 further comprising from about 1% (by total weight of the formulation) to about 10% (by total weight of the formulation) of a humectant.

46. The sunscreen wipe as set forth in claim 27 wherein the humectant is selected from the group consisting of glycerin, sorbitol, polyols, sugars, hydrogenated starch hydrolysates, salts of PCA, lactates, and urea.

47. The sunscreen wipe as set forth in claim 27 further comprising an additional ingredient selected from the group consisting of film formers, sunscreen boosters, vitamins, skin protectants, powders, skin conditioners, botanicals, natural extracts, alpha hydroxy acids, moisturizers, fats and oils, lipids, fatty alcohols, fatty acids, preservatives, chelating agents, antioxidants, colorants, pigments, optical brighteners, essential oils, dry-feel modifiers, and fragrances.

48. The sunscreen wipe as set forth in claim 27 wherein the wipe substrate is comprised of a material selected from the group consisting of meltblown, coform, air-laid, bonded-carded web, hydroentangled, and spunbond.

49. The sunscreen wipe as set forth in claim 48 wherein the material is comprised of synthetic or natural fibers.

50. A sunscreen wipe comprising a wipe substrate and a sunscreen formulation, the sunscreen formulation comprising from about 73% (by total weight of the formulation) to about 73.4% (by total weight of the formulation) water, about 5% (by total weight of the formulation) glycerin, from about 0.1% (by total weight of the formulation) to about 0.5% (by total weight of the formulation) Pemulen TR-2, from about 5% (by total weight of the formulation) to about 20% (by total weight of the formulation) of a high spreadability emollient, from about 7% (by total weight of the formulation) to about 12% (by total weight of the formulation) sunscreen active, from about 0.1% (by total weight of the formulation) to about 5% (by total weight of the formulation) stabilizing emulsifier, and an amount of neutralizer sufficient to adjust the pH of the sunscreen formulation to a range of from about 5.4 to about 5.6, wherein the sunscreen wipe has a transfer rate of at least about 4%.

51. The sunscreen wipe as set forth in claim 50 wherein the sunscreen formulation further comprises a fragrance.

52. The sunscreen wipe as set forth in claim 50 wherein the transfer rate is from at least about 4% to about 15%.

53. The sunscreen wipe as set forth in claim 50 wherein the transfer rate is from about 5% to about 10%.

54. The sunscreen wipe as set forth in claim 50 wherein the sunscreen formulation has a low shear viscosity of from about 1000 cPs to about 10,000 cPs.

55. The sunscreen wipe as set forth in claim 50 wherein the sunscreen formulation has a low shear viscosity of from about 2000 cPs to about 8,000 cPs.

56. The sunscreen wipe as set forth in claim 50 wherein the sunscreen formulation has a low shear viscosity of from about 3000 cPs to about 6,000 cPs.

57. The sunscreen wipe as set forth in claim 50 wherein the sunscreen formulation has a high shear viscosity of no more than about 3000 cPs.

58. The sunscreen wipe as set forth in claim 50 wherein the sunscreen formulation has a high shear viscosity of no more than about 2000 cPs.

59. The sunscreen wipe as set forth in claim 50 wherein the sunscreen formulation has a high shear viscosity of no more than about 1000 cPs.

60. The sunscreen wipe as set forth in claim 50 wherein the sunscreen formulation has a pH of from about 5 to about 6.

61. The sunscreen wipe as set forth in claim 50 wherein the sunscreen formulation has a pH of from about 5.4 to about 5.6.

62. The sunscreen wipe as set forth in claim 50 wherein the sunscreen active is selected from the group consisting of benzophenone-3 (oxybenzone), benzophenone-4 (sulisobenzone), benzophenone-8 (dioxybenzone), butylmethoxydibenzoylmethane (avobenzone), cinoxate, DEA-methoxycinnamate, digalloyl trioleate, 1-(3,4-dimethoxyphenyl)-4,4-4-dimethyl-1,3-pentanediene, ethyl dihydroxypropyl PABA, ethylhexyl dimethyl PABA (padimate 0), ethylhexyl methoxycinnamate (octinozate), ethylhexyl salicylate (octisalate), 4-(2-beta-glucopyranosiloxy) propoxy-2-hydroxybenzophenone, glyceryl PABA, homosalate, mentyl anthranilate, octocrylene, PABA (aminobenzoic acid), phenylbenzimidazole sulfonic acid (ensulizole), red petrolatum, TEA salicylate, titanium dioxide, zinc oxide, surface treated titanium dioxide, surface treated zinc oxide, Spirulina Platensis Powder, Vitis Vinifera (Grape) Seed Extract, Helianthus Annus (Sunflower) Seed Extract, tocopherol, and combinations thereof.

63. The sunscreen wipe as set forth in claim 50 wherein the sunscreen active is encapsulated.

64. The sunscreen wipe as set forth in claim 50 wherein the sunscreen active comprises a UV absorber selected from the group consisting of terephthalidene dicamphor sulfonic acid, drometrizole trisiloxane, benzylylidene malonate polysiloxane, diethylhexylbutamido triazone, methylene-bis-benzotriazolyl tetermethylbutylphenol, disodium pheny dibenzimidazole tetersulfonate, bis-ethylhexyloxyphenol methoxyphenyl triazine, diethylamino hydroxylbenzoly hexyl benzoate, and combinations thereof.

65. The sunscreen wipe as set forth in claim 50 wherein the stabilizing emulsifier has a hydrophilic/lipophilic balance of from about 1 to about 7.

66. The sunscreen wipe as set forth in claim 50 wherein the stabilizing emulsifier is selected from the group consisting of sorbitan monooleate, sorbitan sesquioleate, sorbitan isostearate, sorbitan trioleate, PEG-22/dodecyl glycol copolymer, PEG-45/dodecyl glycol copolymer, polyglyceryl-3-diisostearate, polyglycerol esters of oleic/isostearic acid, polyglyceryl-6 hexaricinolate, polyglyceryl-4 oleate, polyglyceryl-4 oleate/PEG-8 propylene glycol cocoate, oleamide DEA, sodium glyceryl oleate phosphate, hydrogenated vegetable glycerides phosphate, glyceryl stearate, and combinations thereof.

67. The sunscreen wipe as set forth in claim 50 wherein the neutralizing agent is a base.

68. The sunscreen wipe as set forth in claim 50 wherein the neutralizing agent is triethanolamine.

69. The sunscreen wipe as set forth in claim 50 wherein the neutralizing agent is sodium borate.

70. The sunscreen wipe as set forth in claim 50 wherein the high spreadability emollient is selected from the group consisting of dimethicone, cyclomethicone, C12-15 alkyl benzoates, isopropyl palmitate, isopropyl myristate, isopropyl benzoate, diisopropyl adipate, isostearyl benzoate, octyl benzoate, dipropylene glycol dibenzoate, octyldodecyl benzoate, cetearyl octanoate, C12-15 alkyl octanoate, octyl dodecanol, dimethyl capramide, butyloctyl salicylate, diisoamyl malate, and combinations thereof.

71. The sunscreen wipe as set forth in claim 50 further comprising an additional ingredient selected from the group consisting of film formers, sunscreen boosters, vitamins, skin protectants, powders, skin conditioners, botanicals, natural extracts, alpha hydroxy acids, moisturizers, fats and oils, lipids, fatty alcohols, fatty acids, preservatives, chelating agents, antioxidants, colorants, pigments, optical brighteners, essential oils, dry-feel modifiers, and fragrances.

72. The sunscreen wipe as set forth in claim 50 wherein the wipe substrate is comprised of a material selected from the group consisting of meltblown, coform, air-laid, bonded-carded web, hydroentangled, and spunbond.

73. The sunscreen wipe as set forth in claim 72 wherein the material is comprised of synthetic or natural fibers.

74. A sunscreen formulation suitable for use in combination with a wipe substrate, the sunscreen formulation comprising from about 53.5% (by total weight of the formulation) to about 98.7% (by total weight of the formulation) water, from about 1% (by total weight of the formulation) to about 40% (by total weight of the formulation) sunscreen active, from about 0.1% (by total weight of the formulation) to about 0.5% (by total weight of the formulation) Pemulen TR-2, from about 0.1% (by total weight of the formulation) to about 5% (by total weight of the formulation) stabilizing emulsifier, and from about 0.1% (by total weight of the formulation) to about 1% (by total weight of the formulation) neutralizing agent, wherein the sunscreen formulation has a pH of from about 5 to about 6.

75. The sunscreen formulation as set forth in claim 74 wherein the sunscreen formulation has a low shear viscosity of from about 2000 cPs to about 10,000 cPs.

76. The sunscreen formulation as set forth in claim 74 wherein the sunscreen formulation has a high shear viscosity of no more than about 1000 cPs.

77. The sunscreen formulation as set forth in claim 74 wherein the sunscreen formulation has a pH of from about 5.4 to about 5.6.

78. The sunscreen formulation as set forth in claim 74 wherein the sunscreen active is selected from the group consisting of benzophenone-3 (oxybenzone), benzophenone-4 (sulisobenzone), benzophenone-8 (dioxybenzone), butylmethoxydibenzoylmethane (avobenzone), cinoxate, DEA-methoxycinnamate, digalloyl trioleate, 1-(3,4-dimethoxyphenyl)-4,4-4-dimethyl-1,3-pentanediene, ethyl dihydroxypropyl PABA, ethylhexyl dimethyl PABA (padimate 0), ethylhexyl methoxycinnamate (octinozate), ethylhexyl salicylate (octisalate), 4-(2-beta-glucopyranosiloxy) propoxy-2-hydroxybenzophenone, glyceryl PABA, homosalate, mentyl anthranilate, octocrylene, PABA (aminobenzoic acid), phenylbenzimidazole sulfonic acid (ensulizole), red petrolatum, TEA salicylate, titanium dioxide, zinc oxide, surface treated titanium dioxide, surface treated zinc oxide, Spirulina Platensis Powder, Vitis Vinifera (Grape) Seed Extract, Helianthus Annus (Sunflower) Seed Extract, tocopherol, and combinations thereof.

79. The sunscreen formulation as set forth in claim 74 wherein the sunscreen active comprises a UV absorber selected from the group consisting of terephthalidene dicamphor sulfonic acid, drometrizole trisiloxane, benzylylidene malonate polysiloxane, diethylhexylbutamido triazone, methylene-bis-benzotriazolyl tetermethylbutylphenol, disodium phenyl dibenzimidazole tetersulfonate, bis-ethylhexyloxyphenol methoxyphenyl triazine, diethylamino hydroxylbenzoly hexyl benzoate, and combinations thereof.

80. The sunscreen formulation as set forth in claim 74 wherein the neutralizing agent is a base.

81. The sunscreen formulation as set forth in claim 74 wherein the sunscreen formulation further comprises from about 0.5% (by total weight of the formulation) to about 15% (by total weight of the formulation) of a high spreadability emollient.

82. The sunscreen formulation as set forth in claim 81 wherein the high spreadability emollient is selected from the group consisting of dimethicone, cyclomethicone, C12-15 alkyl benzoates, isopropyl palmitate, isopropyl myristate, isopropyl benzoate, diisopropyl adipate, isostearyl benzoate, octyl benzoate, dipropylene glycol dibenzoate, octyldodecyl benzoate, cetearyl octanoate, C12-15 alkyl octanoate, octyl dodecanol, dimethyl capramide, butyloctyl salicylate, diisoamyl malate, and combinations thereof.

83. The sunscreen formulation as set forth in claim 74 further comprising from about 1% (by total weight of the formulation) to about 10% (by total weight of the formulation) of a humectant.

84. The sunscreen formulation as set forth in claim 74 further comprising an additional ingredient selected from the group consisting of film formers, sunscreen boosters, vitamins, skin protectants, powders, skin conditioners, botanicals, natural extracts, alpha hydroxy acids, moisturizers, fats and oils, lipids, fatty alcohols, fatty acids, preservatives, chelating agents, antioxidants, colorants, pigments, optical brighteners, essential oils, dry-feel modifiers, and fragrances.

85. A sunscreen formulation suitable for use in combination with a wipe substrate, the sunscreen formulation comprising from about 53.5% (by total weight of the formulation) to about 98.2% (by total weight of the formulation) water, from about 1% (by total weight of the formulation) to about 40% (by total weight of the formulation) sunscreen active, from about 0.1% (by total weight of the formulation) to about 0.5% (by total weight of the formulation) Pemulen TR-2, from about 0.1% (by total weight of the formulation) to about 5% (by total weight of the formulation) stabilizing emulsifier, and from about 0.1% (by total weight of the formulation) to about 1% (by total weight of the formulation) neutralizing agent, wherein the formulation has a low shear viscosity of from about 1000 cPs to about 20,000 cPs, a high shear viscosity of no more than about 3000 cPs, and a pH of from about 5 to about 6.

86. The sunscreen formulation as set forth in claim 85 wherein the sunscreen formulation has a low shear viscosity of from about 2000 cPs to about 10,000 cPs.

87. The sunscreen formulation as set forth in claim 85 wherein the sunscreen formulation has a low shear viscosity of from about 3000 cPs to about 6,000 cPs.

88. The sunscreen formulation as set forth in claim 85 wherein the sunscreen formulation has a high shear viscosity of no more than about 2000 cPs.

89. The sunscreen formulation as set forth in claim 85 wherein the sunscreen formulation has a high shear viscosity of no more than about 1000 cPs.

90. The sunscreen formulation as set forth in claim 85 wherein the sunscreen formulation has a pH of from about 5.4 to about 5.6.

91. The sunscreen formulation as set forth in claim 85 wherein the sunscreen active is selected from the group consisting of benzophenone-3 (oxybenzone), benzophenone-4 (sulisobenzone), benzophenone-8 (dioxybenzone), butylmethoxydibenzoylmethane (avobenzone), cinoxate, DEA-methoxycinnamate, digalloyl trioleate, 1-(3,4-dimethoxyphenyl)-4,4-4-dimethyl-1,3-pentanediene, ethyl dihydroxypropyl PABA, ethylhexyl dimethyl PABA (padimate 0), ethylhexyl methoxycinnamate (octinozate), ethylhexyl salicylate (octisalate), 4-(2-beta-glucopyranosiloxy) propoxy-2-hydroxybenzophenone, glyceryl PABA, homosalate, mentyl anthranilate, octocrylene, PABA (aminobenzoic acid), phenylbenzimidazole sulfonic acid (ensulizole), red petrolatum, TEA salicylate, titanium dioxide, zinc oxide, surface treated titanium dioxide, surface treated zinc oxide, Spirulina Platensis Powder, Vitis Vinifera (Grape) Seed Extract, Helianthus Annus (Sunflower) Seed Extract, tocopherol, and combinations thereof.

92. The sunscreen formulation as set forth in claim 85 wherein the sunscreen active comprises a UV absorber selected from the group consisting of terephthalidene dicamphor sulfonic acid, drometrizole trisiloxane, benzylylidene malonate polysiloxane, diethylhexylbutamido triazone, methylene-bis-benzotriazolyl tetermethylbutylphenol, disodium pheny dibenzimidazole tetersulfonate, bis-ethylhexyloxyphenol methoxyphenyl triazine, diethylamino hydroxylbenzoly hexyl benzoate, and combinations thereof.

93. The sunscreen formulation as set forth in claim 85 wherein the stabilizing emulsifier is selected from the group consisting of sorbitan monooleate, sorbitan sesquioleate, sorbitan isostearate, sorbitan trioleate, PEG-22/dodecyl glycol copolymer, PEG-45/dodecyl glycol copolymer, polyglyceryl-3-diisostearate, polyglycerol esters of oleic/isostearic acid, polyglyceryl-6 hexaricinolate, polyglyceryl-4 oleate, polyglyceryl-4 oleate/PEG-8 propylene glycol cocoate, oleamide DEA, sodium glyceryl oleate phosphate, hydrogenated vegetable glycerides phosphate, glyceryl stearate, and combinations thereof.

94. The sunscreen formulation as set forth in claim 85 wherein the neutralizing agent is a base.

95. The sunscreen formulation as set forth in claim 85 wherein the sunscreen formulation further comprises from about 0.5% (by total weight of the formulation) to about 15% (by total weight of the formulation) of a high spreadability emollient.

96. The sunscreen formulation as set forth in claim 95 wherein the high spreadability emollient is selected from the group consisting of dimethicone, cyclomethicone, C12-15 alkyl benzoates, isopropyl palmitate, isopropyl myristate, isopropyl benzoate, diisopropyl adipate, isostearyl benzoate, octyl benzoate, dipropylene glycol dibenzoate, octyldodecyl benzoate, cetearyl octanoate, C12-15 alkyl octanoate, octyl dodecanol, dimethyl capramide, butyloctyl salicylate, diisoamyl malate, and combinations thereof.

97. The sunscreen formulation as set forth in claim 85 further comprising from about 1% (by total weight of the formulation) to about 10% (by total weight of the formulation) of a humectant.

98. The sunscreen formulation as set forth in claim 85 further comprising an additional ingredient selected from the group consisting of film formers, sunscreen boosters, vitamins, skin protectants, powders, skin conditioners, botanicals, natural extracts, alpha hydroxy acids, moisturizers, fats and oils, lipids, fatty alcohols, fatty acids, preservatives, chelating agents, antioxidants, colorants, pigments, optical brighteners, essential oils, dry-feel modifiers, and fragrances.

99. A medicinal wipe comprising a wipe substrate and a medicinal formulation, the medicinal formulation comprising from about 44% (by total weight of the formulation) to about 94% (by total weight of the formulation) water, from about 0.1% (by total weight of the formulation) to about 0.5% (by total weight of the formulation) Pemulen TR-2, from about 5% (by total weight of the formulation) to about 10% (by total weight of the formulation) of a high spreadability emollient, from about 0.1% (by total weight of the formulation) to about 50% (by total weight of the formulation) medicinal active, from about 0.1% (by total weight of the formulation) to about 5% (by total weight of the formulation) stabilizing emulsifier, and an amount of neutralizing agent sufficient to adjust the pH of the formulation from about 5.4 to about 5.6, wherein the medicinal wipe has a transfer rate of at least about 4%.

100. The medicinal wipe as set forth in claim 99 wherein the medicinal active is a skin protectant.

101. The medicinal wipe as set forth in claim 100 wherein the skin protectant is selected from the group consisting of calamine, cocoa butter, petrolatum, zinc oxide, lanolin, and witch hazel.

102. The medicinal wipe as set forth in claim 99 wherein the medicinal active is a psoriasis medication.

103. The medicinal wipe as set forth in claim 102 wherein the psoriasis medication is selected from the group consisting of pyrithone zinc, salicylic acid, and selenium sulfide.

104. The medicinal wipe as set forth in claim 99 wherein the medicinal active is an antimicrobial.

105. The medicinal wipe as set forth in claim 104 wherein the antimicrobial is selected from the group consisting of bacitracin, chlortetrcycline hydrochloride, neomycin sulfate, and tetracycline hydrochloride.

106. The medicinal wipe as set forth in claim 99 wherein the medicinal active is an antifungal.

107. The medicinal wipe as set forth in claim 106 wherein the antifungal is selected from the group consisting of cliquinol, haloprogin, miconazole nitrate, povidone iodine, and tolnaftate.

108. The medicinal wipe as set forth in claim 99 wherein the medicinal active is an anorectal drug.

109. The medicinal wipe as set forth in claim 108 wherein the anorectal drug is selected from the group consisting of benzocaine, benzyl alcohol, dibucaine, lidocaine, and tetracaine.

110. The medicinal wipe as set forth in claim 99 wherein the medicinal active is an acne medication.

111. The medicinal wipe as set forth in claim 110 wherein the acne medication is selected from the group consisting of salicylic acid and benzoyl peroxide.
